SEQUENCE LISTING (9 sequences)

SEQ ID NO: 1 - protein, 39 aa, synthetic construct
HGDGSFSDEMNTILDNLAARDFINWLIQTRITDGGGGSK

SEQ ID NO: 2 - protein, 39 aa, synthetic construct
  SITE 2: note = X is Aib
HXDGSFSDEMNTILDNLAARDFINWLIQTRITDGGGGSK

SEQ ID NO: 3 - protein, 44 aa, synthetic construct
HGDGSFSDEMNTILDNLAARDFINWLIQTRITDGGGGSGGGGSK

SEQ ID NO: 4 - protein, 39 aa, synthetic construct
HGDGSFSDEMNTILDNLAARDFINWLIQTHITDGGGGSK

SEQ ID NO: 5 - protein, 33 aa, synthetic construct
HGDGSFSDEMNTILDNLAARDFINWLIQTKITD

SEQ ID NO: 6 - DNA, 117 bp, synthetic construct
  source 1..117: mol_type = other DNA
catggtgatggttctttttctgatgaaatgaatactattcttgataatcttgctgctcgtgattttattaattggcttattcaaactcgtattactgatggtggtggtggttctaaa

SEQ ID NO: 7 - DNA, 111 bp, synthetic construct
  source 1..111: mol_type = other DNA
gatggttctttttctgatgaaatgaatactattcttgataatcttgctgctcgtgattttattaattggcttattcaaactcgtattactgatggtggtggtggttctaaa

SEQ ID NO: 8 - protein, 37 aa, synthetic construct
DGSFSDEMNTILDNLAARDFINWLIQTRITDGGGGSK

SEQ ID NO: 9 - protein, 33 aa, Homo sapiens
HADGSFSDEMNTILDNLAARDFINWLIQTKITD

(19) United States
(12) Patent Application Publication
(10) Pub. No.: US 2024/0150424 A1
(43) Pub. Date: May 9, 2024

(54) DIRECTED CHEMICAL CONJUGATE OF GLUCAGON-LIKE PEPTIDE-2 MUTANT, AND USE THEREOF

(71) Applicant: PEG-BIO BIOPHARM CO.,LTD.(CHONGQING), Chongqing (CN)

(72) Inventors: Kai FAN, Chongqing (CN); Yongliang PENG, Chongqing (CN); Qing CHEN, Chongqing (CN); Qi ZHANG, Chongqing (CN); Yong HE, Chongqing (CN); Xiaolan QIN, Chongqing (CN); Jie SU, Chongqing (CN); Xin MEI, Chongqing (CN); Wenjie ZHAO, Chongqing (CN)

(73) Assignee: PEG-BIO BIOPHARM CO.,LTD.(CHONGQING), Chongqing (CN)

(21) Appl. No.: 18/417,994

(22) Filed: Jan. 19, 2024

Related U.S. Application Data
(63) Continuation of application No. PCT/CN2022/112935, filed on Aug. 17, 2022.

(30) Foreign Application Priority Data
Jul. 20, 2021 (CN) CN202110819379.9

(51) Int. Cl. C07K 14/605; A61K 47/60; A61P 1/04

(52) U.S. Cl. CPC C07K 14/605; A61K 47/60; A61P 1/04

(57) ABSTRACT

A directed chemical conjugate of a glucagon-like peptide-2 mutant, and a use thereof are provided. The glucagon-like peptide-2 mutant is based on the human glucagon-like peptide-2 through multiple site mutations and carboxy terminus extensions. By introducing a targeted chemical modification site, the glucagon-like peptide-2 mutant has biological activities consistent with the natural human glucagon-like peptide-2, and meanwhile, the half-life period in vivo is significantly prolonged, and the administration frequency is reduced, thereby achieving the purpose of treating or preventing short intestinal syndrome, intestinal mucosa injury caused by chemotherapy medicaments or radioactive treatment factors, ulcerative enteritis, chronic enteritis and non-inflammatory bowel injury disease.

### CROSS-REFERENCE TO RELATED APPLICATIONS

This application is a continuation of International Application No. PCT/CN2022/112935, filed on Aug. 17, 2022, which claims priority to and the benefit of Patent Application No. 202110819379.9, filed to China National Intellectual Property Administration on Jul. 20, 2021, which are incorporated herein by reference in their entirety.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jan. 12, 2024, and is approximately 9,562 bytes in size.

### TECHNICAL FIELD

The present disclosure relates to the fields of biotechnology and pharmaceuticals, particularly to a directed chemical conjugate of a glucagon-like peptide-2 mutant, and a use thereof.

### BACKGROUND

Glucagon-like peptide-2 (GLP-2) gene is contained in a gene sequence of proglucagon (PG). The gene of proglucagon is post-translationally expressed as a single-chain precursor protein having 160 amino acids. The precursor protein produces a series of proglucagon-derived peptides (PGDPs) with different biological activities under the action of prohormone convertases (PCs), including glucagon, glicentin relative peptides (GRPP), intervening peptide-1 (IP-1), intervening peptide-2 (IP-2), glucagon-like peptide-1 (GLP-1), and glucagon-like peptide-2 (GLP-2). GLP-2 is located at positions 126 to 158 of the single-chain precursor protein and consists of 33 amino acid residues.

Glucagon-like peptide-2 (GLP-2) is an intestinal hormone secreted by human intestinal L-type cells, which has multiple biological effects in vivo, mainly including (1) stimulating the proliferation of crypt cells in intestinal mucosa and inhibiting their apoptosis to promote the growth of the intestinal mucosa and regenerative repair after injury ((DG Burrin et al., Am J Physiol Gastrointest Liver Physiol 279(6): G1249-1256, 2000)); (2) stimulating the growth of intestinal villi to promote the absorption of nutrients by intestine (PALLE B J et al., Gastrpenterology 120: 806-815, 2001); (3) increasing a thickness of intestinal mucosa and enhancing intestinal barrier functions (Cameron H L et al., Am J Physiol Gastrointest Liver Physiol 284(6): G905-12, 2003); (4) inhibiting gastric acid secretion and gastric motility ((Meier J J et al., Gastrpenterology 130(1): 44-54, 2006); (5) increasing intestinal blood supply (Bremholm L et al., Scand J Gastroenterol. 44(3): 314-9, 2009).

Glucagon-like peptide-2 (GLP-2) exists in an active form of 1-33 sequence in humans, and there is also hydrolyzed GLP-2(3-33), but it has no biological activity. Natural hGLP-2 has an half-life in vivo of 7 min in humans (P B Jeppesen et al., Teduglutide (ALX-0600), a dipeptidyl peptidase IV resistant glucagon-like peptide 2 analogue, improves intestinal function in short bowel syndrome patients) and is readily hydrolyzed by dipeptidyl peptidase IV (DPP-IV) to inactive GLP-2(3-33). The inactive GLP-2(3-33) has a half-life in vivo of only 27 min and is eventually cleared by the kidney through a series of degradation reactions (WENDY TAVARES et al., Enzymatic- and renal-dependent catabolism of the intestinotropic hormone glucagon-like peptide-2 in rats). To solve the problem of the short half-life in vivo, NPS company mutated alanine at position 2 to glycine on the basis of hGLP-2¹⁻³³. The mutated GLP-2 analogue can resist DPP-IV degradation and prolong the half-life in vivo to 2 h. This GLP-2 analogue was named Teduglutide by NPS company, and can be injected subcutaneously once a day at a dose of 0.05 mg/Kg to patients. This medicament was approved by FDA in 2012 for the treatment of short intestinal syndrome in adults and was approved in 2019 for the treatment of short intestinal syndrome in children. The medicament delivery frequency is once a day, which reduces patient compliance. The development of long-acting GLP-2 analogues is urgent.

### SUMMARY

The present disclosure is directed at solving, at least in part, one of the technical problems in the related art. To this end, an object of the present disclosure is to provide a glucagon-like peptide-2 mutant and a conjugate thereof formed by targeted chemical modification. The glucagon-like peptide-2 mutant is based on the human glucagon-like peptide-2 through multiple site mutations and carboxy terminus extensions. By introducing a targeted chemical modification site, the glucagon-like peptide-2 mutant has biological activities consistent with the natural human glucagon-like peptide-2, and meanwhile, the half-life period in vivo is significantly prolonged, and the medicament delivery frequency is reduced, thereby achieving the purpose of treating or preventing short intestinal syndrome, intestinal mucosa injury caused by a chemotherapy medicament or a radioactive treatment factor, ulcerative enteritis, chronic enteritis and non-inflammatory bowel injury disease.

To this end, in one aspect, the present disclosure provides a glucagon-like peptide-2 mutant. According to an embodiment of the present disclosure, compared with an amino acid sequence of wild-type glucagon-like peptide-2, an amino acid at position 30 in an amino acid sequence of the glucagon-like peptide-2 mutant being a basic amino acid other than lysine,
wherein the amino acid sequence of the wild-type glucagon-like peptide-2 is as set forth in SEQ ID NO: 9.

The amino acid sequence of SEQ ID NO: 9 is as follows: HADGS-FSDEM-NTILD-NLAAR-DFINW-LIQTK-ITD.

According to an embodiment of the present disclosure, the glucagon-like peptide-2 mutant further has at least one of the following additional technical features.

According to an embodiment of the present disclosure, the glucagon-like peptide-2 mutant has a p.Lys30Arg or p.Lys30His mutation.

According to an embodiment of the present disclosure, the glucagon-like peptide-2 mutant further has a p.Ala2Gly or p.Ala2Aib mutation.

In view of the problems that the existing hGLP-2 analogues have a short half-life period that affects the drug efficacy and a high administration frequency that reduces patient compliance, the inventors have inventively found that, based on the amino acid sequence of the wild-type glucagon-like peptide-2 set forth in SEQ ID NO: 9, a specific mutation at the amino acid at position 30 does not affect the biological activity of hGLP-2 and is beneficial to prolong the half-life period in vivo of the hGLP-2 mutant. Performing a specific mutation at an amino acid at position 2 on this basis can further prolong the half-life period in vivo of the glucagon-like peptide-2 mutant, thereby improving the drug efficacy.

According to an embodiment of the present disclosure, the amino acid sequence of the glucagon-like peptide-2 mutant is HX₁DGSFSDEMNTILDNLAARDFINWLIQTX₂ITD, and
wherein X₁ is Gly or Aib, and X₂ is Arg or His.

In another aspect, the present disclosure provides a glucagon-like peptide-2 mutant derivative. According to an embodiment of the present disclosure, the glucagon-like peptide-2 mutant derivative includes the glucagon-like peptide-2 mutant, an extended amino acid sequence and lysine (Lys), wherein: a carboxy terminus of the glucagon-like peptide-2 mutant is linked to an amino terminus of the extended amino acid sequence, and a carboxy terminus of the extended amino acid sequence is linked to the lysine.

According to an embodiment of the present disclosure, the glucagon-like peptide-2 mutant derivative further has at least one of the following additional technical features.

According to an embodiment of the present disclosure, the extended amino acid sequence is selected from at least one of (Gₘ₁Sₙ₁)ₓ₁, (Sₙ₂Gₘ₂)ₓ₂, (Gₘ₃Sₙ₃Gₘ₄)ₓ₃, and (Sₙ₄Gₘ₅ Sₙ₅)ₓ₄, and
wherein m1 represents the number of glycines, n1 represents the number of serines, and x1 represents the number of repetitions of (Gₘ₁Sₙ₁) peptide, m1 and n1 are any integer between 1 and 4, x1 is any integer between 1 and 3, and m1+n1=5, preferably, m1=4, n1=1, and x1=1 or 2.

According to an embodiment of the present disclosure, m2 represents the number of glycines, n2 represents the number of serines, and x2 represents the number of repetitions of (Sₙ₂Gₘ₂) peptide, m2 and n2 are any integer between 1 and 4, x2 is any integer between 1 and 3, and m2+n2=5, preferably, x2=1.

According to an embodiment of the present disclosure, m3 and m4 represent the number of glycines, n3 represents the number of serines, and x3 represents the number of repetitions of (Gₘ₃Sₙ₃Gₘ₄) peptide, m3, m4 and n3 are any integer between 1 and 3, x3 is any integer between 1 and 3, and m3+m4+n3=5.

According to an embodiment of the present disclosure, m5 represents the number of glycines, n4 and n5 represent the number of serines, and x4 represents the number of repetitions of (Sₙ₄Gₘ₅Sₙ₅) peptide, m5, n4 and n5 are any integer between 1 and 3, x4 is any integer between 1 and 3, and m5+n4+n5=5.

According to an embodiment of the present disclosure, the amino acid sequence of the glucagon-like peptide-2 mutant derivative is HX₁DGSFSDEMNTILDNLAARDFINWLIQTX₂ITDX₃K, wherein:
X₁ is Gly or Aib;
X₂ is Arg or His;
X₃ (extended amino acid sequence) is (Gₘ₁Sₙ₁)ₓ₁;
m1 represents the number of glycines;
n1 represents the number of serines;
x1 represents the number of repetitions of (Gₘ₁Sₙ₁) peptide, and x1 is any integer between 1 and 3; and
m1 and n1 are any integer between 1 and 4, and m1+n1=5;
preferably, m1=4, n1=1, and x1=1 or 2.

According to an embodiment of the present disclosure, the amino acid sequence of the glucagon-like peptide-2 mutant derivative is as set forth in SEQ ID NO: 1 to SEQ ID NO: 4.

In another aspect, the present disclosure provides a nucleic acid molecule. According to an embodiment of the present disclosure, the nucleic acid molecule encodes the glucagon-like peptide-2 mutant or the glucagon-like peptide-2 mutant derivative.

In another aspect, the present disclosure provides an expression vector. According to an embodiment of the present disclosure, the expression vector includes the nucleic acid molecule.

In another aspect, the present disclosure provides a host cell. According to an embodiment of the present disclosure, the host cell includes the nucleic acid molecule or the expression vector, or expresses the glucagon-like peptide-2 mutant or the glucagon-like peptide-2 mutant derivative.

In another aspect, the present disclosure provides a method for preparing the glucagon-like peptide-2 mutant or the glucagon-like peptide-2 mutant derivative. According to an embodiment of the present disclosure, the method comprises:
culturing the host cell under a condition suitable for expressing the glucagon-like peptide-2 mutant or the glucagon-like peptide-2 mutant derivative to produce the glucagon-like peptide-2 mutant or the glucagon-like peptide-2 mutant derivative.

In another aspect, the present disclosure provides a glucagon-like peptide-2 mutant conjugate or a pharmaceutically acceptable salt thereof. According to an embodiment of the present disclosure, the conjugate comprises the glucagon-like peptide-2 mutant derivative and a coupler,
wherein the Lys at a carboxy terminus of the glucagon-like peptide-2 mutant derivative is linked to the coupler via an amide bond.

The present disclosure provides a glucagon-like peptide-2 mutant conjugate, in which Ala2 and Lys30 of hGLP-2 are mutated, the carboxy terminus is extended by 5-15 amino acids, and lysine is designed at the carboxy terminus of the extended amino acid for directed chemical coupling to form a long-acting glucagon-like peptide-2 mutant conjugate with a longer half-life in vivo.

According to an embodiment of the present disclosure, the glucagon-like peptide-2 mutant conjugate or the pharmaceutically acceptable salt thereof further has at least one of the following additional technical features.

According to an embodiment of the present disclosure, the coupler is selected from at least one of a fatty acid coupler and polyethylene glycol.

According to an embodiment of the present disclosure, the fatty acid coupler includes an aliphatic chain and a linker, the aliphatic chain and the linker being linked, wherein the aliphatic chain has a general structural formula HOOC—(CH₂)ₐ—COOH where a is an integer between 12 and 24, preferably, a is 16 or 18.

According to an embodiment of the present disclosure, the linker has a general structural formula (17-amino-10-oxo-3,6,12,15-tetraoxa-9-azaheptadecanoic acid)_{b}-(γGlu)_{c} where b is 1 or 2 and c is 1 or 2, and preferably, b is 1 and c is 1.

According to an embodiment of the present disclosure, the aliphatic chain and the linker are linked via an amide bond.

According to an embodiment of the present disclosure, the polyethylene glycol has a molecular weight of 5 KDa to 40 KDa, preferably 20 KDa.

According to an embodiment of the present disclosure, the polyethylene glycol has a linear or branched structure, preferably a linear structure.

According to an embodiment of the present disclosure, the polyethylene glycol is polyethylene glycol having an activating group selected from N-hydroxysuccinimide or acid chloride.

In another aspect, the present disclosure provides the use of the glucagon-like peptide-2 mutant, the glucagon-like peptide-2 mutant derivative, and the glucagon-like peptide-2 mutant conjugate or the pharmaceutically acceptable salt thereof in the preparation of a medicament. According to an embodiment of the present disclosure, the medicament is used for treating and/or preventing an intestinal-related disease.

The glucagon-like peptide-2 mutant provided in the present disclosure is a uniquely designed molecular structure, which has the same biological activity as the natural hGLP-2, with the lysine designed at the carboxy terminus extended backwards as a targeted chemical modification site for the coupler. By reversibly binding to albumin, the glucagon-like peptide-2 mutant-fatty acid conjugate provided in the present disclosure has the effect of significantly prolonging the half-life in vivo and reducing the administration frequency to once a week, thereby achieving the purpose of treating or preventing short intestinal syndrome, intestinal mucosa injury caused by chemotherapy medicament or radioactive treatment factor, ulcerative enteritis, chronic enteritis and non-inflammatory bowel injury disease.

According to an embodiment of the present disclosure, the intestinal-related diseases are selected from at least one of short intestinal syndrome, intestinal mucosa injury caused by chemotherapy or radiotherapy, ulcerative enteritis, chronic enteritis and non-inflammatory bowel injury.

The short intestinal syndrome includes, but is not limited to, short intestinal syndrome caused by surgical resection.

In another aspect, the present disclosure provides a pharmaceutical composition. According to an embodiment of the present disclosure, the pharmaceutical composition includes the glucagon-like peptide-2 mutant and/or the glucagon-like peptide-2 mutant derivative and/or the glucagon-like peptide-2 mutant conjugate or the pharmaceutically acceptable salt thereof.

The pharmaceutical composition provided in the present disclosure has the effects of stimulating the growth of intestinal mucosa, preventing bowel injury, promoting repair after intestinal mucosa injury, and treating intestinal-related diseases.

According to an embodiment of the present disclosure, the pharmaceutical composition further includes at least one of a buffer salt, an excipient and a protective agent.

According to an embodiment of the present disclosure, the buffer salts are selected from at least one of acetates, phosphates, borates and carbonates, and preferably, phosphates.

According to an embodiment of the present disclosure, the excipients are selected from at least one of mannitol, sucrose, maltose and trehalose, and preferably, mannitol and/or trehalose.

According to an embodiment of the present disclosure, the protective agents include, but are not limited to, His, Gly, Ala and Arg, and preferably, His.

According to an embodiment of the present disclosure, the pharmaceutical composition further includes another medicament for treating or preventing an intestinal-related disease or a medicament for cancer chemotherapy and/or radiotherapy.

According to an embodiment of the present disclosure, a dosage form of the pharmaceutical composition is selected from injections, pills, lyophilized powders, tablets, capsules or granules.

According to an embodiment of the present disclosure, the pharmaceutical composition provided in the present disclosure is administered to a patient having intestinal-related diseases by subcutaneous injection at a dose of 0.01 to 0.5 mg/Kg once a week.

In another aspect, the present disclosure provides a pharmaceutical single dosage form. According to an embodiment of the present disclosure, the pharmaceutical single dosage form includes 0.1 to 10 mg of the glucagon-like peptide-2 mutant, the glucagon-like peptide-2 mutant derivative, or the glucagon-like peptide-2 mutant conjugate or the pharmaceutically acceptable salt thereof.

In another aspect, the present disclosure provides a method for treating and/or preventing an intestinal-related disease. According to an embodiment of the present disclosure, the method includes administering to a subject suffering or suspected of having the intestinal-related disease at least one of:
the glucagon-like peptide-2 mutant as described above;
the glucagon-like peptide-2 mutant derivative as described above;
the nucleic acid molecule as described above;
the expression vector as described above;
the host cell as described above;
the glucagon-like peptide-2 mutant conjugate or the pharmaceutically acceptable salt thereof as described above;
the pharmaceutical composition as described above; and
the pharmaceutical single dosage form as described above.

According to an embodiment of the present disclosure, the intestinal-related disease is selected from at least one of short intestinal syndrome, intestinal mucosa injury caused by chemotherapy or radiotherapy, ulcerative enteritis, chronic enteritis, and non-inflammatory bowel injury.

In another aspect, the present disclosure provides the use of the glucagon-like peptide-2 mutant as described above, the glucagon-like peptide-2 mutant derivative as described above, the nucleic acid molecule as described above, the expression vector as described above, the host cell as described above, the glucagon-like peptide-2 mutant conjugate or the pharmaceutically acceptable salt thereof as described above, the pharmaceutical composition as described above, or the pharmaceutical single dosage form as described above in the treatment and/or prevention of an intestinal-related disease.

According to an embodiment of the present disclosure, the intestinal-related disease is selected from at least one of short intestinal syndrome, intestinal mucosa injury caused by chemotherapy or radiotherapy, ulcerative enteritis, chronic enteritis, and non-inflammatory bowel injury.

Additional aspects and advantages of the present disclosure will be set forth in part in the description that follows and, in part, will be obvious from the description or may be learned by practice of the present disclosure.

### BRIEF DESCRIPTION OF DRAWINGS

The above and/or additional aspects and advantages of the present disclosure will become apparent and readily appreciated from the following description of the embodiments, taken in conjunction with the accompanying drawings, in which:

FIG. 1 shows the LC-MS detection results of PT01;

FIG. 2 shows the LC-MS detection results of PF01;

FIG. 3 shows the LC-MS detection results of PT02;

FIG. 4 shows the peptide map detection results of PF01;

FIG. 5 shows the peptide map detection results of PF04;

FIG. 6 shows the in vitro cell viability of Teduglutide, PT01, PT02, PT03 and PT04 glucagon-like peptide-2 mutants;

FIG. 7 shows the changes in CIT (citrulline) of Teduglutide (Tedu), PF01 and PF04 in the animal model of intestinal chemotherapy injury, with the Model as the control group;

FIG. 8 shows case sections for a pharmacodynamic study of Teduglutide (Ted), PF01 and PF04 on the intestinal disease caused by 5-FU;

FIG. 9 shows a pharmacodynamic study of PF01 on the intestinal disease caused by indomethacin-body weight;

FIG. 10 shows a pharmacodynamic study of PF01 on the intestinal disease caused by indomethacin-serum citrulline content;

FIG. 11 shows a pharmacodynamic study of PF01 on the intestinal disease caused by indomethacin-statistical results of α-acidic protein content in serum;

FIG. 12 shows a pharmacodynamic study of PF01 on the intestinal disease caused by indomethacin-statistical results of α-acidic protein content in the supernatant of small intestinal tissues, wherein No Indo refers to a control group without indomethacin injection;

FIG. 13 shows the pharmacokinetic drug-time curve of PF01 in Cynomolgus macaques;

FIG. 14 shows the chemical structure of an octadecyl fatty acid coupler used in Example 2;

FIG. 15 shows the structure of a polyethylene glycol coupler used in Example 2.

### DESCRIPTION OF EMBODIMENTS

The examples of the present disclosure will be described in detail below. The examples described below are exemplary and are merely intended to explain the present disclosure but should not be construed as a limitation to the present disclosure.

In addition, the terms “first” and “second” are merely intended for the purpose of description but should not be construed as an indication or implication of relative importance or an implicit indication of the number of the indicated technical features. Therefore, a feature defined by “first” or “second” may explicitly or implicitly include at least one such feature. In the description of the present disclosure, the “plurality” means at least two, for example, two or three, unless otherwise specifically defined.

According to some specific embodiments of the present disclosure, the present disclosure provides a glucagon-like peptide-2 mutant conjugate. The amino acid sequence of natural GLP-2 is engineered by the inventors and chemically coupled to the modifiers with different molecular weights and structures to form candidate coupling compounds with various structures. Specifically, Ala2 and Lys30 of hGLP-2 are mutated, the carboxy terminus is extended by 5 to 15 amino acids, and lysine is designed at the carboxy terminus of the extended amino acid for directed chemical coupling to form a long-acting glucagon-like peptide-2 mutant conjugate with a longer half-life in vivo. The present disclosure further provides a conjugate directly chemically coupled to fatty acid and a pharmaceutical composition thereof, and their use for treating short intestinal syndrome, intestinal mucosa injury caused by chemotherapy or radiotherapy, and ulcerative enteritis, chronic enteritis and non-inflammatory bowel injury.

According to a specific embodiment of the present disclosure, the chemical conjugate of the glucagon-like peptide-2 mutant provided in the present disclosure includes a glucagon-like peptide-2 mutant and a coupler directed chemically conjugated thereto.

The glucagon-like peptide-2 mutant has the same biological activity as the natural hGLP-2 receptor.

According to a specific embodiment of the present disclosure, the glucagon-like peptide-2 mutant is mutated on the basis of hGLP-2, mutations sites include Ala2 and Lys30, extension of amino acids at carboxy terminus, and design of Lys at the carboxy terminus of the extended peptide. The mutation at the Ala2 site may be a natural or non-natural amino acid, and preferably, Gly or Aib. The Lys30 site is mutated to another basic amino acid other than lysine, and preferably, Arg. The Lys added at the carboxy terminus of the extended peptide is used for directed chemical coupling.

According to a specific embodiment of the present disclosure, the number of extended amino acids at the carboxy terminus of Asp33 is 5 to 15, and the extended amino acids conform to (Gₘ₁Sₙ₁)ₓ₁, (Sₙ₂Gₘ₂)ₓ₂, (Gₘ₃Sₙ₃Gₘ₄)ₓ₃, or (Sₙ₄Gₘ₅Sₙ₅)ₓ₄.

According to a specific embodiment of the present disclosure, in the structure of (Gₘ₁Sₙ₁)ₓ₁, m1 represents the number of glycines, n1 represents the number of serines, and x1 represents the number of repetitions of the (Gₘ₁Sₙ₁) peptide, m1 and n1 are any integer between 1 and 4, x1 is any integer between 1 and 3, and m1+n1=5, preferably, m1=4, n1=1, and x1=1 or 2.

According to a specific embodiment of the present disclosure, in the structure of (Sₙ₂Gₘ₂)ₓ₂, m2 represents the number of glycines, n2 represents the number of serines, and x2 represents the number of repetitions of the (Sₙ₂Gₘ₂) peptide, m2 and n2 are any integer between 1 and 4, x2 is any integer between 1 and 3, and m2+n2=5, preferably, x2=1.

According to a specific embodiment of the present disclosure, in the structure of (Gₘ₃Sₙ₃Gₘ₄)ₓ₃, m3 and m4 represent the number of glycines, n3 represents the number of serines, and x3 represents the number of repetitions of the (Gₘ₃Sₙ₃Gₘ₄) peptide, m3, m4 and n3 are any integer between 1 and 3, x3 is any integer between 1 and 3, and m3+m4+n3=5.

According to a specific embodiment of the present disclosure, in the structure of (Sₙ₄Gₘ₅ Sₙ₅)ₓ₄, m5 represents the number of glycines, n4 and n5 represent the number of serines, and x4 represents the number of repetitions of the (Sₙ₄Gₘ₅Sₙ₅) peptide, m5, n4 and n5 are any integer between 1 and 3, x4 is any integer between 1 and 3, and m5+n4+n5=5.

According to a specific embodiment of the present disclosure, the coupler used for the glucagon-like peptide-2 mutant may be a fatty acid coupler or a polyethylene glycol coupler.

According to a specific embodiment of the present disclosure, the fatty acid coupler includes an aliphatic chain and a linker, wherein the aliphatic chain conforms to the structure of HOOC—(CH₂)ₐ—COOH where a is an integer between 12 and 24 and represents the number of repetitions of the (CH₂) group, preferably, 16 or 18; the linker conforms to the structure of (17-amino-10-oxo-3,6,12,15-tetraoxa-9-azaheptadecanoic acid)_{b}-(γGlu)_{c} where b is an integer 1 or 2 and represents the number of 17-amino-10-oxo-3,6,12,15-tetraoxa-9-azaheptadecanoic acid in series, and preferably 1; c is an integer 1 or 2 and represents the number of γGlu in series, and preferably 1.

The Glu-α-amino group of the linker is coupled to a carboxyl group of the aliphatic chain, the Glu-γ-carboxyl group is coupled to an amino group of 17-amino-10-oxo-3,6,12,15-tetraoxa-9-azaheptadecanoic acid, and a carboxyl group of 17-amino-10-oxo-3,6,12,15-tetraoxa-9-azaheptadecanoic acid is coupled to the F-amino group of lysine at the carboxy terminus of the glucagon-like peptide-2 mutant. The coupling of the linker to the aliphatic chain may be one-to-one or two-to-one, and the coupling of the glucagon-like peptide-2 mutant to the fatty acid coupler may be one-to-one or two-to-one, and preferably, one-to-one.

The fatty acid coupler is subjected to directed chemical coupling to Lys at the carboxy terminus of the glucagon-like peptide-2 mutant provided in the present disclosure via an amide bond formation to form the glucagon-like peptide-2 mutant conjugate. The activating group for the coupling of the γGlu to 17-amino-10-oxo-3,6,12,15-tetraoxa-9-azaheptadecanoic acid is preferably N-hydroxysuccinimide or acyl chloride. The activating group for the coupling of the γGlu to the fatty acid is preferably N-hydroxysuccinimide or acyl chloride. The activating group for coupling the fatty acid coupler to the glucagon-like peptide-2 mutant is preferably N-hydroxysuccinimide or acyl chloride.

According to a specific embodiment of the present disclosure, the polyethylene glycol has a linear or branched structure, and preferably, a linear structure, and a molecular weight of 5 KDa to 40 KDa, and preferably, 20 KDa. The activating group for the coupling of the polyethylene glycol to the glucagon-like peptide-2 mutant is preferably N-hydroxysuccinimide or acid chloride.

The glucagon-like peptide-2 mutant has the same biological activity as the natural human glucagon-like peptide-2.

According to a specific embodiment of the present disclosure, the pharmaceutical composition including the glucagon-like peptide-2 mutant conjugate includes, but is not limited to, the glucagon-like peptide-2 mutant conjugate, a buffer salt including, but not limited to, acetates, phosphates, borates and carbonates, and preferably, phosphates, an excipient including, but not limited to, mannitol, sucrose, maltose and trehalose, and preferably, mannitol and trehalose, and a protective agent including, but not limited to, His, Gly, Ala and Arg, and preferably, His.

According to other specific embodiments of the present disclosure, the pharmaceutical composition provided in the present disclosure further includes pharmaceutically acceptable carriers, including any solvent, solid excipient, diluent, binder, disintegrant, or other liquid excipient, dispersing agent, flavoring or suspending agent, surface active agent, isotonic agent, thickening agent, emulsifying agent, preservative, solid binder, glidant or lubricant, and the like, suitable for the particular desired dosage form.

According to some specific embodiments of the present disclosure, the glucagon-like peptide-2 mutant, the glucagon-like peptide-2 mutant derivative and the glucagon-like peptide-2 mutant conjugate of the present disclosure may be incorporated into pharmaceutical compositions suitable for parenteral administration (e.g., intravenous, subcutaneous, intraperitoneal, and intramuscular administration). These pharmaceutical compositions may be prepared into various forms, such as liquid, semi-solid and solid dosage forms, including, but not limited to, liquid solutions (e.g., injection and infusion solutions), dispersing agents or suspending agents, tablets, pills, powders, liposomes and suppositories.

According to a specific embodiment of the present disclosure, the pharmaceutical composition may be formulated as an injection or a lyophilized powder, and preferably, a lyophilized powder. The lyophilized powder may be redissolved for intravenous, intramuscular, or subcutaneous injection, and preferably, subcutaneous injection.

According to a specific embodiment of the present disclosure, the glucagon-like peptide-2 mutant conjugate has the effects of stimulating the growth of intestinal mucosa, preventing metabolic bowel injury, and promoting and protecting repair after intestinal mucosa injury. The mucosa injury disease includes, but are not limited to, intestinal mucosa injury caused by surgical resection, intestinal mucosa injury caused by chemotherapy or radiotherapy, and ulcerative enteritis. The pharmaceutical composition of the glucagon-like peptide-2 mutant conjugate has a longer half-life in vivo.

The inventors have found that engineered GLP-2 mutant polypeptides, PT01, PT02, PT03 and PT04, have in vitro activities consistent with the natural human glucagon-like peptide-2, and candidate compounds formed by chemical coupling to the coupler have an enhanced in vivo half-life, thereby significantly prolonging the duration of action of the candidate compounds in vivo. Specifically, hGLP-2 has an in vivo half-life of 7 min in humans; Teduglutide has an in vivo half-life of 0.5 h in rats and 2 h in humans; PF04 has an in vivo half-life of 19 h in rats; and PF01 has an in vivo half-life of 21 h in rats and 30 h in cynomolgus monkeys.

The amino acid sequence of PT01 (SEQ ID NO: 1):
HGDGSFSDEMNTILDNLAARDFINWLIQTRITDGGGGSK.
The amino acid sequence of PT02 (SEQ ID NO: 2):
HAibDGS-FSDEM-NTILD-NLAAR-DFINW-LIQTR-ITD-GGGGSK.
The amino acid sequence of PT03 (SEQ ID NO: 3):
HGDGSFSDEMNTILDNLAARDFINWLIQTRITDGGGGSGGGGSK.
The amino acid sequence of PT04 (SEQ ID NO: 4):
HGDGSFSDEMNTILDNLAARDFINWLIQTHITDGGGGSK.

According to a specific embodiment of the present disclosure, the pharmaceutical composition provided in the present disclosure is administered to a patient having an intestinal-related disease by subcutaneous injection at a dose of 0.01 mg to 0.5 mg/Kg once a week.

The chemical coupling products of the glucagon-like peptide-2 mutant of the present disclosure to fatty acid have the effect of stimulating the growth of intestinal mucosa and promoting repair after intestinal mucosa injury after administration, and have a significant drug efficacy and a significant dose-response relationship in the dose range of 0.01 mg to 0.5 mg/Kg.

### Definitions

The “glucagon-like peptide-2 mutant” of the present disclosure is obtained by mutating the natural glucagon-like peptide-2 through a genetic engineering technology. The term “analogue” refers to at least 50% sequence homology to the natural glucagon-like peptide-2. The term “natural glucagon-like peptide-2” refers to human glucagon-like peptide-2, the amino acid sequence of which is HADGSFSDEMNTILDNLAARDFINWLIQTKITD (SEQ ID NO: 9). The term “mutation” refers to the alteration, deletion, insertion and addition of a certain gene sequence fragment by a genetic engineering technology. The term “genetic engineering technology” is a technique well known to those of ordinary skill in the art.

The “mutation” and “engineering” of the present disclosure may be used interchangeably.

The “non-natural amino acid” of the present disclosure refers to an amino acid that cannot be expressed by recombination.

The “Aib” of the present disclosure refers to 2-aminoisobutyric acid, a non-natural amino acid.

The “non-basic amino acid” of the present disclosure refers to an amino acid having no other amino group other than an α-amino group or being unable to ionize NH4⁺;

The terms “targeted chemical modification,” “chemical modification,” “modification,” “directed chemical coupling,” “chemical coupling,” and “coupling” may be used interchangeably in the present disclosure and refer to covalent attachment of the carboxyl group of a coupler to the F-amino group of lysine. The term “modifier” refers to a polymer used for proteins and polypeptide drug modification, such as fatty acid, polyethylene glycol, polypeptide derivative, and the like.

It is well known that fatty acids have one carboxyl group at each end, one of which is normally blocked with t-butanol, and the other is used for activation. Activated fatty acid refers to a fatty acid derivative with a functional group (or an activating group), which is currently mainly used for proteins and polypeptide drug modification.

The fatty acid used for the targeted chemical modification of the glucagon-like peptide-2 mutant in the present disclosure is a fatty acid with an activating group. The activating group of the fatty acid includes, but is not limited to, N-hydroxysuccinimide, N-acyl urea, acid chloride, active ester, or other highly chemically reactive groups. In a particular embodiment, the activating group of the fatty acid is N-hydroxysuccinimide.

The “coupling of the glucagon-like peptide-2 mutant to the fatty acid coupler may be one-to-one or two-to-one, and preferably, one-to-one” of the present disclosure, wherein “one-to-one” refers to covalent attachment of one glucagon-like peptide-2 mutant to one fatty acid coupler, and wherein “two-to-one” refers to covalent attachment of two glucagon-like peptide-2 mutant to one fatty acid coupler.

The “candidate compound,” “coupling product,” and “conjugate” of the present disclosure may be used interchangeably and refer to a product obtained by coupling the glucagon-like peptide-2 mutant to a modifier.

The “naked peptide” of the present disclosure refers to a polypeptide or protein that has not been modified.

The “octadecyl fatty acid modifier,” “octadecyl modifier,” and “18C” of the present disclosure may be used interchangeably and mean that the carbon chain backbone is octadecane.

The “hexadecyl fatty acid modifier,” “hexadecyl modifier,” and “16C” of the present disclosure may be used interchangeably and mean that the carbon chain backbone is hexadecane.

The “tetradecyl fatty acid modifier,” “tetradecyl modifier,” and “14C” of the present disclosure may be used interchangeably and mean that the carbon chain backbone is tetradecane.

The “dipeptide” of the present disclosure refers to a His-Aib peptide, wherein the His represents histidine and the Aib represents 2-aminoisobutyric acid.

The terms “treating” and “preventing” used herein, and words derived therefrom, do not necessarily imply 100% or complete treatment or prevention. Rather, there are varying degrees of treatment or prevention that would be considered by one of ordinary skill in the art to be of potential benefit or therapeutic effect. Moreover, the treatment or prevention provided in the present disclosure may include a disease being treated or prevented. Additionally, for purposes herein, the “preventing” may encompass delaying the onset of a disease, or symptoms or conditions thereof.

Unless otherwise defined, all technical and scientific terms used in the present disclosure have the same meaning as understood by one of ordinary skill in the art. The practitioner may refer to Current Protocols in Molecular Biology (Ausubel) for definitions and terms in the art. Abbreviations for amino acid residues are standard three-letter or single-letter codes among the 20 commonly used L-type amino acids well known as one of ordinary skill in the art.

The directed chemical coupling products of the glucagon-like peptide-2 mutant of the present disclosure may be used, alone or in combination, for treating an intestinal disease with reduced serum citrulline content. The intestinal disease includes, but is not limited to, short intestinal syndrome, bowel injury caused by chemotherapy or radiotherapy, enteritis caused by intestinal insufficiency, and the like.

The glucagon-like peptide-2 mutant of the present disclosure is used as a medicament for treating enteritis caused by chemotherapy in patients representative of model animals, children, or adults. In a specific embodiment, the conjugate may be used alone or in combination, having a significant enhancement of small bowel function.

The directed chemical coupling products of the glucagon-like peptide-2 mutant of the present disclosure can be used as combined drug for parenteral nutritional support, with the effect of improving intestinal function and enhancing intestinal absorption.

The present disclosure is further illustrated by the following examples, but any example or combination thereof should not be construed as limiting the scope or embodiments of the present disclosure. The scope of the present disclosure is defined by the appended claims, which will be apparent to one of ordinary skill in the art in combination with this description and general knowledge in the art. In addition, publications or patents are cited in the present disclosure for the purpose of more clearly describing the present disclosure, the entire contents of which are incorporated herein by reference as if repeated herein in their entirety.

The glucagon-like peptide-2 mutant of the present disclosure may be prepared by recombinant expression using strains including, but not limited to, E. coli and yeast, chemical synthesis, or a combination of both.

The examples of the present disclosure will be described in detail below. The examples described below are exemplary and are merely intended to explain the present disclosure but should not be construed as a limitation to the present disclosure.

Examples, where specific technologies or conditions are not specified, are implemented in accordance with technologies or conditions described in the literature in the art or according to the product description. All used agents or instruments, whose manufacturers are not specified, are conventional commercially available products.

### Example 1: Preparation of Glucagon-Like Peptide-2 Mutants

Preparation of PT01: the nucleic acid sequence of SEQ ID NO: 6 was designed based on the amino acid sequence as set forth in SEQ ID NO: 1 and Escherichia cob preferred codons and constructed into an expression vector pet-9a plasmid (SEQ ID NO: 6 encodes the amino acid sequence of SEQ ID NO: 1). The constructed correct expression plasmid was transformed into Escherichia cob host strains, and screened to obtain recombinant expression strains. The recombinant strains were inoculated into a YT culture medium, induced by IPTG, and then fermented and cultured. After the fermentation solution was centrifuged, the bacteria were collected and stored at −20° C. for later use.

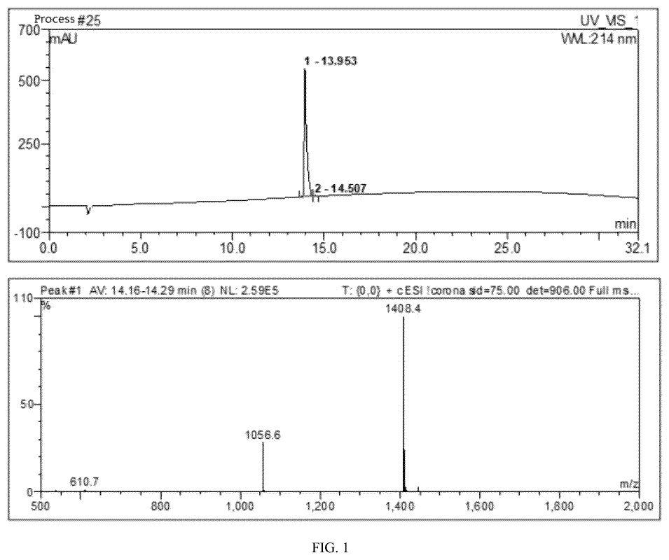

The fermented bacteria were lysed, separated and concentrated by a series of purification chromatography columns and methods to obtain a PT01 protein. Briefly, PT01 was adsorbed on an ion exchange resin chromatography column and eluted with different concentrations of salts. The eluted sample was collected, purified by a reverse phase chromatography column, and linearly gradient eluted by gradually increasing the organic solvent content in the mobile phase. An effluent containing the PT01 sample was collected and freeze-dried to obtain a PT01 raw material. The purity of the PT01 raw material evaluated by HPLC was greater than 98%, and the molecular weight and amino acid sequence of the PT01 raw material verified by LC-MS were the same as the theoretical values (as shown in FIG. 1).

Reference was made to the preparation of PT01 for both the preparation of PT03 and PT04.

Preparation of PT02: an expression vector plasmid was constructed using the nucleic acid sequence of SEQ ID NO: 7 (SEQ ID NO: 7 encodes the amino acid sequence shown in SEQ ID NO: 8). The constructed correct expression plasmid was transformed into Escherichia cob expression host strains, and screened to obtain recombinant expression strains. The recombinant strains were inoculated into a YT culture medium, induced by IPTG, and then fermented and cultured. After centrifugation, the bacteria were collected and stored at −20° C. for later use.

The fermented bacteria were lysed, separated and concentrated by a series of purification chromatography columns and methods to obtain a PT02 pre-protein. Briefly, the PT02 pre-protein was adsorbed on an ion exchange resin chromatography column and eluted with different concentrations of salts. The eluted sample was collected, purified by a reverse phase chromatography column, and linearly gradient eluted by gradually increasing the organic solvent content in the mobile phase. An effluent containing the PT02 pre-protein was collected and freeze-dried to obtain a PT02 pre-protein dry powder. The purity of the PT02 pre-protein determined by HPLC was greater than 98%, and the molecular weight and amino acid sequence of the PT02 pre-protein verified by LC-MS were the same as the theoretical values. To a carbonate buffer with a buffer ion strength of 20 to 200 mmol/L, a modified pH of 9.0 to 11.0, and a protein concentration of 5.0 to 25.0 mg/ml, dipeptide (His-Aib peptide, purchased from Chengdu Pukang Biotechnology Co. Ltd.) was added at a molar ratio of PT02 pre-protein:dipeptide=1:1.2 and stirred at room temperature for 1 to 3 h. The free PT02 pre-protein and free dipeptide were removed by reverse phase silica gel column chromatography, and then a PT02 raw material was obtained after freeze drying. The purity of the PT02 raw material evaluated by HPLC was greater than 98%, and the molecular weight of the PT02 raw material verified by LC-MS was the same as the theoretical value (as shown in FIG. 3).

Table 1 below shows the amino acid sequences of some glucagon-like peptide-2 mutants of the present disclosure and Teduglutide.

| Name | Amino acid sequence | SEQ ID NO: |
|---|---|---|
| PT01 | HGDGS-FSDEM-NTILD-NLAAR-DFINW-LIQTR- | SEQ ID NO: 1 |
| | ITD-GGGGSK | |
| PT02 | HAibDGS-FSDEM-NTILD-NLAAR-DFINW-LIQTR- | SEQ ID NO: 2 |
| | ITD-GGGGSK | |
| PT03 | HGDGS-FSDEM-NTILD-NLAAR-DFINW-LIQTR- | SEQ ID NO: 3 |
| | ITD-GGGGS-GGGGSK | |
| PT04 | HGDGS-FSDEM-NTILD-NLAAR-DFINW-LIQTH- | SEQ ID NO: 4 |
| | ITD-GGGGSK | |
| Tedu- | HGDGS-FSDEM-NTILD-NLAAR-DFINW-LIQTK- | SEQ ID NO: 5 |
| glutide | ITD | |

The nucleic acid sequence of SEQ ID NO: 6 (encoding the amino acid sequence of SEQ ID NO: 1) is as follows:

5′-CATGGTGATGGTTCTTTTTCTGATGAAATGAATACTATTCTTGAT
AATCTTGCTGCTCGTGATTTTATTAATTGGCTTATTCAAACTCGTATT
ACTGATGGTGGTGGTGGTTCTAAA-3′.

The nucleic acid sequence of SEQ ID NO: 7 (encoding the amino acid sequence of SEQ ID NO: 8) is as follows:

5′-GATGGTTCTTTTTCTGATGAAATGAATACTATTCTTGATAATCTTG
CTGCTCGTGATTTTATTAATTGGCTTATTCAAACTCGTATTACTGATGG
TGGTGGTGGTTCTAAA-3′.

The amino acid sequence of SEQ ID NO: 8 is as follows: DGSFSDEMNTILDNLAARDFINWLIQTRITDGGGGSK.

### Example 2: Preparation of Glucagon-Like Peptide-2 Mutant Conjugates

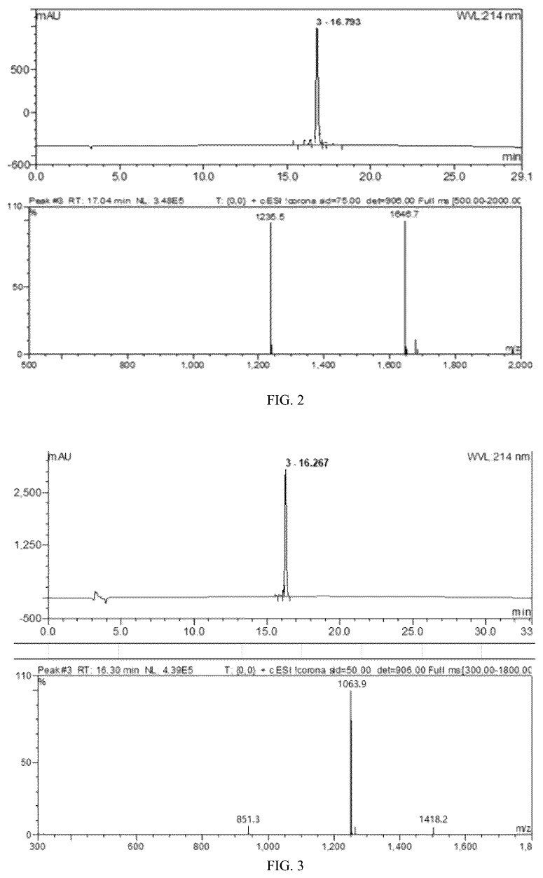

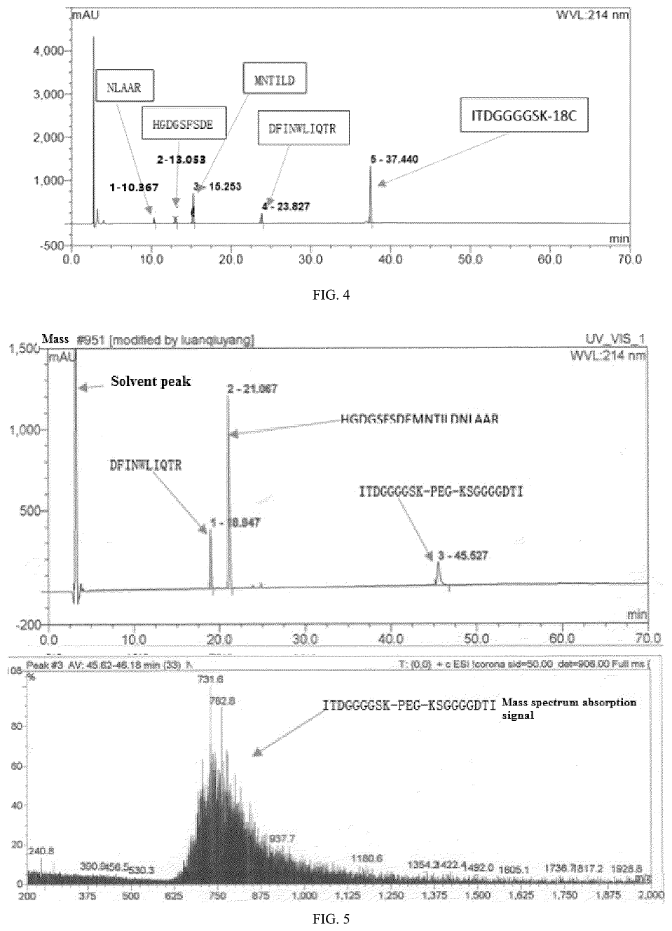

Preparation of PF01: 100 mg of the PT01 raw material (produced by Chongqing Paijin Biotechnology Co. Ltd.) was weighed in a 100 ml beaker and dissolved in 10 ml of 0.1 mol/L carbonate solution at pH 10.5. Then, 540 mg of an octadecyl fatty acid coupler (purchased from Chengdu Pukang Biotechnology Co. Ltd., the structure as shown in FIG. 14) was added at a molar ratio of PT01:octadecyl fatty acid coupler=1:1.2 and stirred at 25° C. for 1 h. The obtained mixture was adjusted to pH 4.5 with 1 mol/L HCl solution to terminate the reaction and centrifuged to collect precipitates. The precipitates were added with an appropriate amount of trifluoroacetic acid, stirred for 1 to 2 h, and dissolved in Tris buffer. The obtained mixture was loaded onto an ion exchange chromatography column and eluted with different concentrations of salts. The eluted sample was collected and finely purified by a reverse phase chromatography column. The purity of the sample detected by RP-HPLC was >98%, and the molecular weight of the target product identified by LC-MS was consistent with the theoretical value (FIG. 2). PF01 was digested by Trypsin protease and V8 enzyme and identified by LC-MS that the coupling site of the octadecyl fatty acid coupler was lysine at the carboxy terminus (peptide map detection results of PF01 in FIG. 4). The PF01 sample was freeze-dried at a low temperature, collected and stored below −15° C. for later use.

Reference was made to the preparation of PF01 for both the preparation of PF02 and PF03.

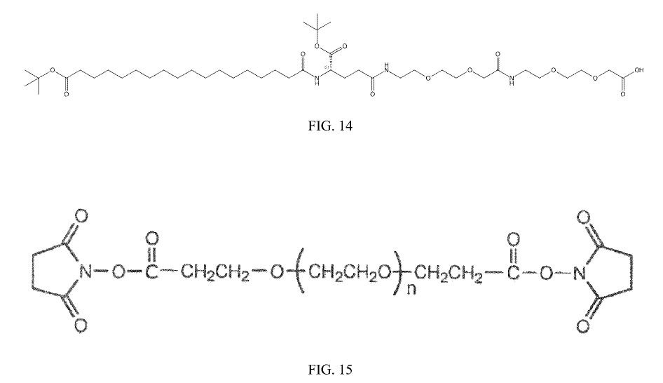

Preparation of PF04: 100 mg of the PT01 raw material (produced by Chongqing Paijin Biotechnology Co. Ltd.) was weighed in a 100 ml beaker and dissolved in 10 ml of 0.1 mol/L carbonate solution at pH 9.5. Then, 568 mg of a polyethylene glycol coupler (purchased from Xiamen Sainuo Bangge Technology Co. Ltd., the structure as shown in FIG. 15) was added at a molar ratio of PT01:polyethylene glycol coupler=1:1.2 and stirred at 25° C. for 1 h. The obtained mixture was adjusted to pH 4.5 with 1 mol/L HCl solution to terminate the reaction and the obtained mixture was adjusted to pH 7.5 with 1 mol/L Tris buffer, loaded onto an ion exchange chromatography column, and eluted with different concentrations of salts. The eluted sample was collected and purified by a molecular sieve chromatography column. The purity of the sample detected by RP-HPLC was >98%. PF04 was digested by Trypsin protease and identified by LC-MS that the coupling site of the polyethylene glycol coupler was lysine at the carboxy terminus (peptide map detection results of PF04 in FIG. 5). The PF04 sample was freeze-dried at a low temperature. The dry powder was collected and stored below −15° C. for later use.

TABLE 2
Determination of the molecular weights of glucagon-
like peptide-2 mutant PT01 and mutant conjugates

| | | Theoretical | Determined | |
|---|---|---|---|---|
| | | molecular | molecular | Coupling |
| Name | Structure | weight | weight | site |
| PT01 | SEQ ID NO: 1 | 4223.60 | 4223.51 | — |
| PF01 | PT01 + octadecyl fatty | 4939.60 | 4938.00 | Lys |
| | acid coupler | | | |
| PF02 | PT01 + hexadecyl fatty | 4590.60 | 4549.26 | Lys |
| | acid coupler | | | |
| PF03 | PT01 + tetradecyl fatty | 4434.04 | 4434.97 | Lys |
| | acid coupler | | | |
| PF04 | PT01 + polyethylene | 28411.20 | — | Lys |
| | glycol coupler | | | |

The LC-MS identification results in Table 2 above show that the determined molecular weights of PT01, PF01, PF02, PF03 and PF04 were basically consistent with the theoretical values and that PT01 of PF01, PF02, PF03 and PF04 was coupled to the coupler via a Lys coupling site.

### Example 3: In Vitro Cell Viability Assay for Glucagon-Like Peptide-2 Mutants

HEK-293-GLP-2R cells (HEK-293 cells were purchased from National Collection of Authenticated Cell Cultures, GLP-2R receptor gene sequence was recombinantly constructed by Shanghai Shengbo Biomedical Technology Co. Ltd., and HEK-293-GLP-2R cells were obtained by transfection and screening by Chongqing Paijin Biotechnology Co. Ltd.) were used to determine the in vitro activities of glucagon-like peptide-2 mutants. The glucagon-like peptide-2 mutants, PT01, PT02, PT03 and PT04, prepared in Example 1 and Teduglutide (NPS company) were diluted to 500 ng/ml with a diluent containing 1 mM IBMX, respectively, and diluted by 3-fold ratio to 8 gradients, and then transferred to cell culture wells containing well-grown recipient cells. The cells were incubated at 37° C., 5% CO₂ for 15 minutes, and then added with a cell lysis buffer. The cell lysates were extracted, and the OD value of cAMP was determined using a cAMP assay kit (RD company). The EC50 values of Teduglutide and the glucagon-like peptide-2 mutants were calculated using an “Origin pro 2017” parametric regression software to detect their in vitro cell viability. The results of the in vitro cell viability assay for each sample are shown in Table 3.

TABLE 3
In vitro cell viability of glucagon-like peptide-2 mutants

| Sample | | |
|---|---|---|
| name | Amino acid sequence | EC50 |
| Teduglutide | HGDGSFSDEMNTILDNLAARDFINWLIQTKITD | 100% |
| PT01 | HGDGSFSDEMNTILDNLAARDFINWLIQTRITDGGGGSK | 125% |
| PT02 | HAibDGSFSDEMNTILDNLAARDFINWLIQTRITDGGGGSK | 91% |
| PT03 | HGDGSFSDEMNTILDNLAARDFINWLIQTRITDGGGGSGGGGSK | 118% |
| PT04 | HGDGSFSDEMNTILDNLAARDFINWLIQTHITDGGGGSK | 109% |

As can be seen from the results in Table 3, the in vitro cell viabilities of PT01, PT03 and PT04 were 105%-130% of that of Teduglutide, taking the in vitro cell viability of Teduglutide as a standard. The in vitro cell viability of PT02 was comparable to that of Teduglutide.

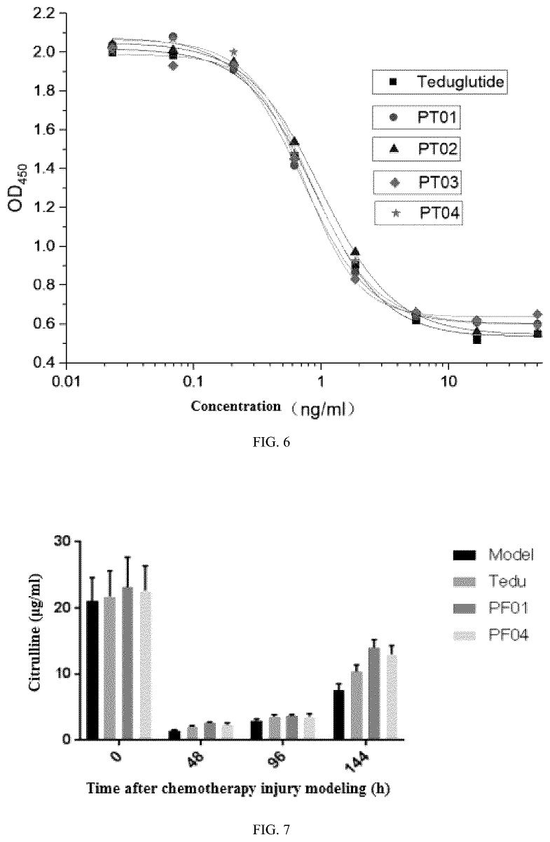

FIG. 6 shows EC50 curves of Teduglutide, PT01, PT02, PT03 and PT04, the activity trends shown in the curves were basically consistent with the trends exhibited by the activity data in Table 3.

### Example 4: Preparation of Glucagon-Like Peptide-2 Mutant Conjugate Lyophilized Powder Formulations

PF01 formulation: a phosphate buffer solution was prepared, and His protective agent and mannitol excipient were added and stirred for dissolution. The concentration of phosphate was 35 mmol/L, the concentration of His was 50 mmol/L, and the content of mannitol was 3%. 100 mg of the PF01 raw material prepared in Example 2 was weighed in a 100 ml clean beaker, added with the phosphate solution containing His and mannitol, and stirred for dissolution. The pH of the solution was adjusted to 7.4±0.2 with HCl or NaOH. The content of the protein was determined. The solution was sterile-filtered, dispensed into vials, and freeze-dried at a low temperature to obtain a PF01 formulation. The PF01 formulation was capped, labeled, and stored at 8° C. below for later use.

Reference was made to the preparation of the PF01 formulation for the preparation of a PF04 formulation (PF04 was prepared using the preparation method of Example 2).

### Example 5: Test of PF01 and PF04 on Promoting Small Intestinal Hyperplasia

Teduglutide (purchased from NPS company), the PF01 formulation, the PF04 formulation and a placebo were used in a mouse experiment to evaluate the effect of PF01 on promoting small intestinal hyperplasia. Forty minor Kunming mice weighing 10 to 20 g were equally divided into 3 groups, 10 mice each group. The control group was subcutaneously administered with the placebo once a day for 9 days, the Ted group was subcutaneously administered at 1 mg/Kg once a day for 9 days, and the PF01 group and the PF04 group were subcutaneously administered at 3 mg/Kg once every three days for a total of 3 times. All experimental animals were weighed before administration. On the 10th day after administration, the body weight was weighed, blood was collected, and small intestine length and weight were determined.

TABLE 4
Experimental results of PF01 and PF04 on promoting
small intestine hyperplasia in minor mice

| | | Body weight | | | |
|---|---|---|---|---|---|
| | Body weight | of mice at | Serum | Small | Small |
| Experimental | of mice before | the end of the | citrulline | intestine | intestine |
| groups | administration | experiment | content | length | weight |
| Control | 13.27 ± 0.52 g | 17.97 ± 0.65 g | 13.00 μg/ml | 34.6 cm | 1.54 g |
| group | | | | | |
| Ted | 13.32 ± 0.49 g | 18.06 ± 0.49 g | 14.47 μg/ml | 37.4 cm | 1.75 g |
| PF01 | 13.13 ± 0.57 g | 19.12 ± 0.56 g | 17.86 μg/ml | 38.3 cm | 2.46 g |
| PF04 | 13.24 ± 0.48 g | 18.64 ± 0.52 g | 16.57 μg/ml | 38.1 cm | 2.05 g |

As shown in Table 4, both the PF01 group and the PF04 group were superior to the Teduglutide group in terms of mice body weight, serum citrulline, small intestine length and small intestine weight, in which PF01 was the best, and all three administration groups were superior to the control group.

### Example 6: Pharmacodynamic Study of PF01 and PF04 on an Intestinal Disease Caused by 5-FU

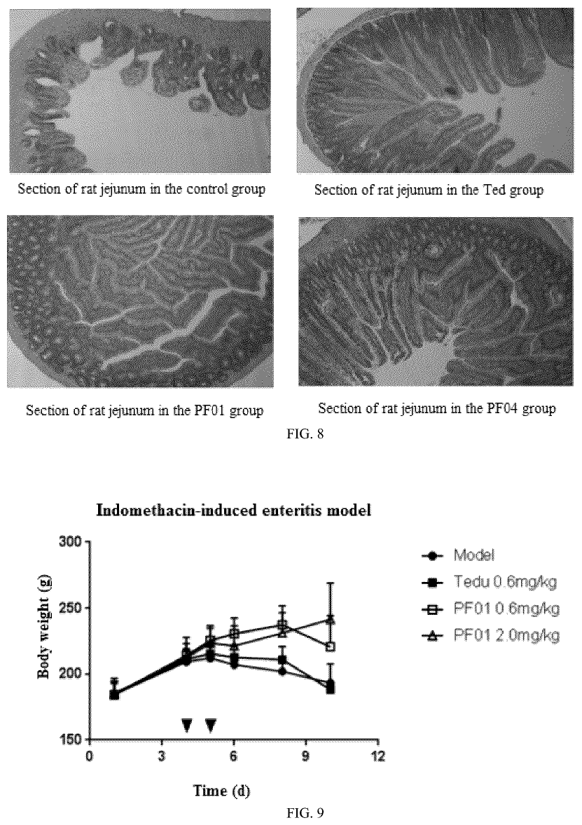

Teduglutide (purchased from NPS company), PF01, PF04 and a placebo were used in a rat experiment to evaluate the efficacy of PF01 and PF04 in diarrhea and intestinal mucosa injury model casused by 5-FU in rats. Forty adult SD rats weighing 200 to 300 g were equally divided into 4 groups, 10 rats each group. A Model group was subcutaneously administered with the placebo once a day for 9 days, a Ted group was subcutaneously administered at 1 mg/Kg once a day for 9 days, and a PF01 group and a PF04 group were subcutaneously administered at 3 mg/Kg once every three days for a total of 3 times. All rats were intraperitoneally injected with 5-FU at 50 mg/Kg once a day on the 4^{th} day to the 6^{th} day. Diarrhea and body weight changes in rats were recorded every day after the first administration of 5-FU. On the 10th day, body weight was weighed, blood was collected, and jejunum was taken for case sections. The statistical data for body weight are shown in Table 5, and the case sections are shown in FIG. 8.

Diarrhea was identified based on scores, with 0 indicating no diarrhea, 1 indicating mild diarrhea with feces adhering to the anus, 2 indicating moderate diarrhea with feces adhering to the hind legs and tail, 3 indicating severe diarrhea with feces adhering to the front legs and abdomen, and 4 indicating death. According to the experimental records, the statistical results are shown in Table 5.

TABLE 5
Efficacy evaluation of PF01 and PF04 in rat models

| | Test items at different times | | | | | |
|---|---|---|---|---|---|---|
| | | | | | | Serum |
| | Body | Body | Body | Body | Body | citrulline |
| | weight | weight 24 h | weight 48 h | weight 72 h | weight 96 h | content 96 h |
| | before the first | after the first | after the first | after the first | after the first | after the first |
| | administration | administration | administration | administration | administration | administration |
| Groups | of 5-FU | of 5-FU | of 5-FU | of 5-FU | of 5-FU | of 5-FU |
| Model group | 262.29 g | 266.01 g | 257.81 g | 259.61 g | 265.41 g | 12.28 μg/ml |
| Ted group | 272.46 g | 274.36 g | 257.94 g | 260.95 g | 275.43 g | 16.50 μg/ml |
| PF01 group | 269.69 g | 281.11 g | 269.85 g | 275.50 g | 287.50 g | 17.99 μg/ml |
| PF04 group | 270.45 g | 278.25 g | 264.62 g | 271.81 g | 282.17 g | 17.38 μg/ml |

TABLE 6
Statistics of diarrhea scores for each group

Time after the administration of 5-FU

| Groups | 24 h | 48 h | 72 h | 96 h |
|---|---|---|---|---|
| Model group | 3/3 | 5/5 | 7/6 | 6/4 |
| Ted group | 0/0 | 4/4 | 4/3 | 3/3 |
| PF01 group | 0/0 | 3/3 | 2/2 | 0/0 |
| PF04 group | 0/0 | 3/3 | 2/2 | 0/0 |

In Table 6, the numerator represents the diarrhea score, and the denominator represents the number of animals with diarrhea.

TABLE 7
Serum citrulline content for each group

| | Time | | | | |
|---|---|---|---|---|---|
| | Before the | 48 h after the | 96 h after the | 144 h after the | |
| | administration | administration | administration | administration | |
| Groups | of 5-FU | of 5-FU | of 5-FU | of 5-FU | |
| model group | 21.05 μg/ml | 1.46 μg/ml | 2.99 μg/ml | 7.52 | μg/ml |
| Ted group | 21.74 μg/ml | 2.04 μg/ml | 3.53 μg/ml | 10.37 | μg/ml |
| PF01 group | 23.13 μg/ml | 2.57 μg/ml | 3.62 μg/ml | 14.02 | μg/ml |
| PF04 group | 22.65 μg/ml | 2.28 μg/ml | 3.58 μg/ml | 12.98 | μg/ml |

FIG. 7 shows the changes in CIT of Teduglutide, PF01 and PF04 in animal intestinal chemotherapy injury models. The change trends before chemotherapy, 48 h after chemotherapy and 144 h after chemotherapy were consistent, i.e. citrulline content in the PF01 group>that in the PF04 group>that in the Ted group>that in the Model group; and 96 h after chemotherapy, the citrulline content in the PF01 group was basically consistent with that in the PF04 group and the Ted group, and all higher than that in the Model group.

The above experimental results showed that in the animal intestinal chemotherapy injury models established using 5-FU, with the process of molding and administration, both PF01 and PF04 could significantly reduce the weight loss, diarrhea and decreased serum citrulline caused by modeling, and accelerate the recovery of body weight and serum citrulline levels in the model rats after modeling. The above pharmacodynamic effects were significantly superior to the Ted group, and PF01 was the best. The test results correspond to the results of the case sections (FIG. 8) determined based on the height of villi and the number of crypt cells: the PF01 group>the PF04 group>the Ted group>the control group.

Based on the determination of body weight, diarrhea, serum citrulline content and case sections in rats, PF01 was superior to Ted in the prevention or treatment of 5-FU-induced intestinal diseases, and PF01 had a lower administration frequency. The results showed that PF01 had a significant therapeutic effect in the prevention or treatment of diarrhea and intestinal mucosa injury caused by chemotherapy medicaments.

### Example 7: Pharmacodynamic Study of PF01 on Indomethacin-Induced Intestinal Diseases

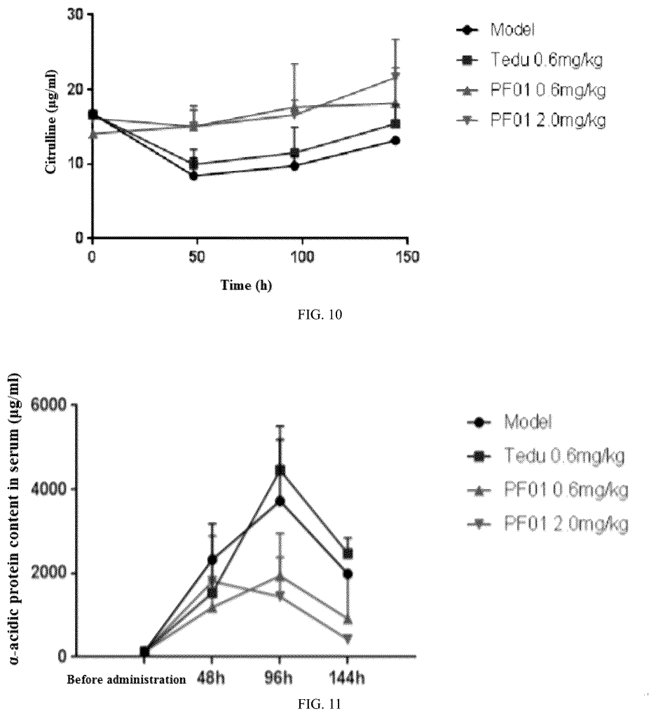

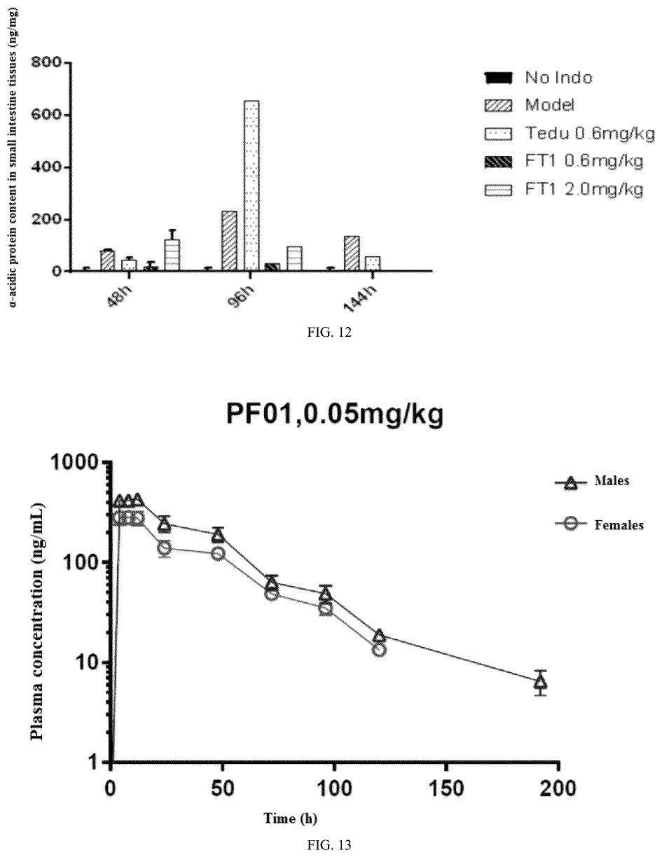

Teduglutide (purchased from NPS company), PF01, and a placebo were used in a rat experiment to evaluate the efficacy of PF01 in indomethacin-induced diarrhea and intestinal mucosa injury models in rats. Forty adult SD rats weighing 200-300 g were equally divided into 4 groups, 10 rats each group. A Model group, a Ted group, a PF1 low dose group and a PF1 high dose group were set. The model group was subcutaneously administered with an equal volume of PBS once a day for 12 days, the Ted group was subcutaneously administered at 0.6 mg/Kg once a day for 12 days, the PF01 low dose group was administered at 0.6 mg/Kg once every three days for a total of 4 times, and the PF01 high dose group was administered at 2.0 mg/Kg once every three days for a total of 4 times. The rats in all groups were injected with indomethacin intraperitoneally at 7 mg/kg once a day on the 4^{th} day to the 5^{th} day. The body weight changes in rats were recorded every day after the first administration of indomethacin. On the 12^{th} day, the body weight was weighed, blood was collected, jejunum tissues were taken, and the serum citrulline content, the α-acidic protein content in serum and the α-acidic protein content in the supernatant of small intestine tissues were determined. The body weight changes are shown in FIG. 9. The statistical results of the serum citrulline content are shown in FIG. 10. The statistical results of the α-acidic protein (α1-AGP) content in serum are shown in FIG. 11. The statistical results of the α-acidic protein content in the supernatant of small intestine tissues are shown in FIG. 12.

The experimental results showed that in the animal intestinal inflammation models established using indomethacin, with the process of molding and administration, PF01 could significantly alleviate the weight loss, increased α-acidic protein content in intestinal tissues and serum and decreased serum citrulline caused by modeling, and accelerate the recovery of body weight and serum citrulline levels in the model rats after modeling. The above pharmacodynamic effects were significantly superior to the Ted group.

Based on the determination of body weight, serum citrulline content, α-acidic protein content in serum and α-acidic protein content in the supernatant of small intestinal tissues in rats, PF01 was superior to Ted in the prevention or treatment of indomethacin-induced intestinal diseases, and PF01 had a lower administration frequency. The results showed that PF01 had a significant therapeutic effect in the treatment of medicament-induced intestinal diseases such as ulcerative enteritis.

### Example 8: Pharmacokinetic Study of PF01 and PF04 in Rats

10 adult SD rats weighing 200-300 g were equally divided into 2 groups, 5 rats each group. PF01 and PF04 were administered subcutaneously at a single dose of 3 mg/Kg, respectively. Serum samples of all rats were collected before administration and 8 h, 24 h, 48 h, 72 h and 96 h after administration, and plasma concentrations were determined by ELISA.

The Tmax of PF01 was basically consistent with that of PF04 in rats. The T_{1/2} of PF01 and PF04 in rats was 20.28 h and 17.63 h, respectively. The Cmax of PF01 and PF04 in rats was 67.48 μg/ml and 57.28 μg/ml, respectively.

### Example 9: Pharmacokinetic Study of PF01 in Cynomolgus Macaques

4 adult Cynomolgus macaques, half males and half females, were used. PF01 was administered by subcutaneous injection at a single dose of 0.05 mg/Kg. Blood samples were collected before administration and 4 h, 8 h, 12 h, 24 h, 48 h, 72 h, 96 h, 120 h, 192 h and 288 h after administration. The concentrations of PF01 in the samples were determined by LC-MS/MS. The plasma concentration data were analyzed using WinNonlin, a pharmacokinetic data analysis software. FIG. 13 shows the pharmacokinetic drug-time curves of PF01 in Cynomolgus macaques. The t_{1/2}, Cmax, Tmax, AUC, Cl and Vd were calculated using a non-compartmental model (NCA).

The experimental results showed that the average values of T_{1/2}, Cmax and Tmax of PF01 in Cynomolgus macaques were 30.92 h, 458.91 ng/ml and 12 h, respectively; the average value of Cl was 2.27 ml/h/kg; the average value of Vd was 101.13 ml/kg; and the AUC was 21.71 h-μg/ml. Based on T_{1/2}, the in vivo half-life of the PF01 medicament was approximately 100 times longer than that of the natural hGLP-2. The glucagon-like peptide-2 mutant conjugates provided in the present disclosure significantly prolonged the in vivo half-life of the glucagon-like peptide-2 in animals.

In the description of this specification, reference to the term “an embodiment,” “some embodiments,” “an example,” “a specific example,” or “some examples,” or the like means that a specific feature, structure, material or characteristic described in combination with the embodiment(s) or example(s) are included in at least one embodiment or example of the present disclosure. In this specification, the illustrative expressions of the above terms do not necessarily refer to the same embodiment or example. Moreover, the specific feature, structure, material, or characteristic described may be combined in any suitable manner in any one or more embodiments or examples. In addition, without mutual contradiction, those skilled in the art may incorporate and combine different embodiments or examples and features of the different embodiments or examples described in this specification.

Although the embodiments of the present disclosure have been illustrated and described above, it should be understood that the above embodiments are illustrative and cannot be construed as limitations on the present disclosure. Various changes, modifications, replacements and variants may be made by those skilled in the above embodiments in the art without departing from the scope of the present disclosure.

What is claimed is:

1. A glucagon-like peptide-2 mutant, compared with an amino acid sequence of wild-type glucagon-like peptide-2, an amino acid at position 30 in an amino acid sequence of the glucagon-like peptide-2 mutant being a basic amino acid other than lysine,
wherein the amino acid sequence of the wild-type glucagon-like peptide-2 is as set forth in SEQ ID NO: 9.

2. The glucagon-like peptide-2 mutant according to claim 1, wherein the glucagon-like peptide-2 mutant has a p.Lys30Arg or p.Lys30His mutation.

3. The glucagon-like peptide-2 mutant according to claim 1, wherein the glucagon-like peptide-2 mutant further has a p.Ala2Gly or p.Ala2Aib mutation.

4. The glucagon-like peptide-2 mutant according to claim 1, wherein the amino acid sequence of the glucagon-like peptide-2 mutant is HX₁DGSFSDEMNTILDNLAARDFINWLIQTX₂ITD, and
wherein X₁ is Gly or Aib, and X₂ is Arg or His.

5. A glucagon-like peptide-2 mutant derivative, comprising the glucagon-like peptide-2 mutant according to claim 1, an extended amino acid sequence, and lysine (Lys), wherein:
a carboxy terminus of the glucagon-like peptide-2 mutant is linked to an amino terminus of the extended amino acid sequence, and a carboxy terminus of the extended amino acid sequence is linked to the lysine.

6. The glucagon-like peptide-2 mutant derivative according to claim 5, wherein the extended amino acid sequence is selected from at least one of (Gₘ₁Sₙ₁)ₓ₁, (Sₙ₂Gₘ₂)ₓ₂, (Gₘ₃Sₙ₃Gₘ₄)ₓ₃, and (Sₙ₄Gₘ₅Sₙ₅)ₓ₄, and
wherein m1 represents the number of glycines, n1 represents the number of serines, and x1 represents the number of repetitions of (Gₘ₁Sₙ₁) peptide, m1 and n1 are any integer between 1 and 4, x1 is any integer between 1 and 3, and m1+n1=5, preferably, m1=4, n1=1, and x1=1 or 2;
optionally, m2 represents the number of glycines, n2 represents the number of serines, and x2 represents the number of repetitions of (Sₙ₂Gₘ₂) peptide, m2 and n2 are any integer between 1 and 4, x2 is any integer between 1 and 3, and m2+n2=5, preferably, x2=1;
optionally, m3 and m4 represent the number of glycines, n3 represents the number of serines, and x3 represents the number of repetitions of (Gₘ₃Sₙ₃Gₘ₄) peptide, m3, m4 and n3 are any integer between 1 and 3, x3 is any integer between 1 and 3, and m3+m4+n3=5; and
optionally, m5 represents the number of glycines, n4 and n5 represent the number of serines, and x4 represents the number of repetitions of (Sₙ₄Gₘ₅Sₙ₅) peptide, m5, n4 and n5 are any integer between 1 and 3, x4 is any integer between 1 and 3, and m5+n4+n5=5.

7. The glucagon-like peptide-2 mutant derivative according to claim 6, wherein the amino acid sequence of the glucagon-like peptide-2 mutant derivative is HX₁DGSFSDEMNTILDNLAARDFINWLIQTX₂ITDX₃K, wherein:
X₁ is Gly or Aib;
X₂ is Arg or His;
X₃ is (Gₘ₁Sₙ₁)ₓ₁;
m1 represents the number of glycines;
n1 represents the number of serines;
x1 represents the number of repetitions of (Gₘ₁Sₙ₁) peptide, and x1 is any integer between 1 and 3; and
m1 and n1 are any integer between 1 and 4, and m1+n1=5;
preferably, m1=4, n1=1, and x1=1 or 2.

8. The glucagon-like peptide-2 mutant derivative according to claim 5, wherein the amino acid sequence of the glucagon-like peptide-2 mutant derivative is as set forth in SEQ ID NO: 1 to SEQ ID NO: 4.

9. A nucleic acid molecule, encoding the glucagon-like peptide-2 mutant derivative according to claim 8.

10. A glucagon-like peptide-2 mutant conjugate or a pharmaceutically acceptable salt thereof, comprising:
the glucagon-like peptide-2 mutant derivative according to claim 8; and
a coupler,
wherein the Lys at a carboxy terminus of the glucagon-like peptide-2 mutant derivative is linked to the coupler via an amide bond.

11. The glucagon-like peptide-2 mutant conjugate or the pharmaceutically acceptable salt thereof according to claim 10, wherein the coupler is selected from at least one of a fatty acid coupler and polyethylene glycol.

12. The glucagon-like peptide-2 mutant conjugate or the pharmaceutically acceptable salt thereof according to claim 11, wherein:
the fatty acid coupler comprises an aliphatic chain and a linker, the aliphatic chain and the linker being linked;
the aliphatic chain has a general structural formula HOOC—(CH₂)ₐ—COOH where a is an integer between 12 and 24, and preferably, a is 16 or 18;
optionally, the linker has a general structural formula (17-amino-10-oxo-3,6,12,15-tetraoxa-9-azaheptadecanoic acid)_{b}-(γGlu)_{c} where b is 1 or 2 and c is 1 or 2, and preferably, b is 1 and c is 1; and
optionally, the aliphatic chain and the linker are linked via an amide bond.

13. The glucagon-like peptide-2 mutant conjugate or the pharmaceutically acceptable salt thereof according to claim 11, wherein:
the polyethylene glycol has a molecular weight of 5 KDa to 40 KDa,
optionally, the polyethylene glycol has a linear or branched structure, preferably, a linear structure,
optionally, the polyethylene glycol is polyethylene glycol having an activating group selected from N-hydroxysuccinimide or acid chloride.

14. A pharmaceutical composition, comprising:
the glucagon-like peptide-2 mutant conjugate or the pharmaceutically acceptable salt thereof according to claim 10 and at least one of a buffer salt, an excipient, and a protective agent, wherein:
optionally, the buffer salt is selected from at least one of acetates, phosphates, borates and carbonates, and preferably, phosphates,
optionally, the excipient is selected from at least one of mannitol, sucrose, maltose and trehalose, and preferably, mannitol and/or trehalose,
optionally, the protective agent comprises His, Gly, Ala and Arg, and preferably, His.

15. The pharmaceutical composition according to claim 14, further comprising another medicament for treating or preventing an intestinal-related disease or a medicament for cancer chemotherapy and/or radiotherapy.

16. The pharmaceutical composition according to claim 14, wherein a dosage form of the pharmaceutical composition is selected from injections, pills, lyophilized powders, tablets, capsules or granules.

17. A pharmaceutical single dosage form, comprising 0.1 to 10 mg of the glucagon-like peptide-2 mutant conjugate or the pharmaceutically acceptable salt thereof according to claim 10.

18. A method for treating and/or preventing an intestinal-related disease, comprising administering to a subject suffering or suspected of having the intestinal-related disease:
the pharmaceutical composition according to claim 14.

19. A method for treating and/or preventing an intestinal-related disease, comprising administering to a subject suffering or suspected of having the intestinal-related disease:
the pharmaceutical single dosage form according to claim 17.

20. The method according to claim 18, wherein the intestinal-related disease is selected from at least one of short intestinal syndrome, intestinal mucosa injury caused by chemotherapy or radiotherapy, ulcerative enteritis, chronic enteritis, and non-inflammatory bowel injury.
